(19) United States
(12) Patent Application Publication
(10) Pub. No.: US 2012/0172698 A1
(43) Pub. Date: Jul. 5, 2012

(54) IMAGING SYSTEM

(75) Inventors: Tat-Jin Teo, Sunnyvale, CA (US); Roger Hastings, Maple Grove, MN (US)

(73) Assignee: BOSTON SCIENTIFIC SCIMED, INC., Maple Grove, MN (US)

(21) Appl. No.: 13/316,862

(22) Filed: Dec. 12, 2011

Related U.S. Application Data
(60) Provisional application No. 61/428,563, filed on Dec. 30, 2010.

(51) Int. Cl. A61B 6/00

(52) U.S. Cl. 600/407

(57) ABSTRACT

Techniques are described that combine intravascular ultrasound (“IVUS”) and optical coherence tomography into a single catheter that utilizes a micro-motor driven imaging core for imaging of patient tissue, e.g., a blood vessel wall. In one example, an imaging assembly includes a catheter, an imaging core with a micro-motor, one or more transducer conductors, an optical coherence tomography apparatus, and one or more optical fibers in communication with the optical coherence tomography apparatus.

### CROSS REFERENCE TO RELATED APPLICATIONS

This application claims the benefit of U.S. Provisional Application No. 61/428,563, entitled, “IMAGING SYSTEM,” by Tat-Jin Teo and Roger Hastings, and filed on Dec. 30, 2010, the entire contents of which being incorporated herein by reference.

### TECHNICAL FIELD

This disclosure relates to medical devices and, more particularly to intravascular imaging devices.

### BACKGROUND

Intravascular ultrasound (“IVUS”) imaging systems provide visual indicia to a practitioner when diagnosing and treating various diseases and disorders. For example, IVUS imaging systems have been used to diagnose blocked blood vessels and to provide information to a practitioner in selecting and placing stents and other devices to restore or increase blood flow to a vessel. IVUS imaging systems have also been used to diagnose plaque build-up in the blood vessels and other intravascular obstructions. IVUS imaging systems can also be used to monitor one or more heart chambers. IVUS imaging systems are often used to visualize various portions of the vascular system that may be difficult to visualize using other imaging techniques, such as angiography, where movement caused by a beating heart or obstruction by one or more structures such as blood vessels can impair the quality of the image retrieved.

An IVUS imaging system can include a control unit, a catheter, and one or more transducers disposed in the catheter. The catheter is configured and arranged for percutaneous insertion into a patient and can be positioned in a lumen or cavity at or near a region to be imaged, such as a blood vessel wall. Electrical pulses generated by the control unit are delivered to the transducer(s) and transformed into acoustic pulses that are transmitted through the blood vessel wall or other patient tissue. Reflected pulses of the transmitted acoustic pulses are absorbed by the transducer(s) and transformed into electrical signals that are converted to an image visible by the practitioner.

Optical Coherence Tomography (“OCT”) is a type of optical coherence-domain reflectometry that uses low coherence interferometry to perform high resolution ranging and cross-sectional imaging. In OCT systems, a light beam from a low coherence light source is split into a reference light beam and a sample light beam. A diffraction grating may be used to provide an optical path difference in one or both light beams. The sample light beam is directed onto a sample and the light scattered from the sample is combined with the reference light beam. The combination of the sample and reference light beams results in an interference pattern corresponding to the variation in the sample reflection with the depth of the sample, along the sample beam. The sample beam typically suffers a high loss of energy due to its interaction with the sample. The reference beam serves as a local oscillator to amplify the interference pattern to a detectable level and therefore must have a much higher energy level than the sample light beam. The interference pattern is detected by a photo detector, whose output is processed to generate a cross-sectional image of the sample. High resolution (e.g., less than 10 micrometer) imaging of the cross-sections of the sample by OCT is useful in biological and medical examinations and procedures, as well as in materials and manufacturing applications.

### SUMMARY

In general, this disclosure describes techniques for intravascular imaging. In particular, this disclosure describes techniques that combine intravascular ultrasound (“IVUS”) and Optical Coherence Tomography (“OCT”) into a single catheter that utilizes a micro-motor driven imaging core for imaging of patient tissue, e.g., a blood vessel wall.

In one example, the disclosure is directed to an imaging assembly. The imaging assembly comprises a catheter having a distal end and a proximal end, the catheter defining a catheter lumen extending from the proximal end to the distal end of the catheter, the catheter configured and arranged for insertion into the vasculature of a patient, an imaging core having a distal end and a proximal end, the imaging core disposed in the distal end of the catheter lumen. The imaging core comprises a drive shaft that extends from the proximal end of the imaging core toward the distal end of the imaging core, the drive shaft defining a shaft lumen, a rotatable magnet disposed about the shaft lumen, the magnet configured and arranged to be rotatable by a magnetic field, a reflective surface configured and arranged to rotate with the magnet, and at least one transducer engaged to the drive shaft and positioned proximal to the reflective surface, the at least one transducer configured and arranged for transforming applied electrical signals to acoustic signals that are transmitted toward the reflective surface and also for transforming received echo signals to electrical signals, the at least one transducer defining a transducer lumen. The assembly further comprises at least one transducer conductor electrically coupled to the at least one transducer and extending to the proximal end of the catheter, an optical coherence tomography (OCT) apparatus configured to transmit an optical beam toward a tissue of the patient and receive reflected light from the tissue, and at least one optical fiber in communication with the OCT apparatus that extends through the shaft lumen from the proximal end of the catheter toward the distal end of the imaging core through the transducer lumen, the optical fiber configured to guide the optical beam.

In another example, the disclosure is directed to an imaging system. The imaging system comprises an imaging assembly such as described in paragraph [0006] above, a user interface, and a control unit coupled to the imaging core. The control unit comprises a pulse generator electrically coupled to the at least one transducer via the at least one transducer conductor, the pulse generator configured and arranged to generate electric signals that are applied to the at least one transducer during a scan, and a processor electrically coupled to the OCT apparatus and the pulse generator. The processor is configured to control generation of the electric signals that are applied to the at least one transducer by the pulse generator, control transmission of the optical beam, via the OCT apparatus, and delivery of the electric signals generated by the pulse generator to the at least one transducer, process the received reflected light, via the optical coherence tomography circuitry, and the received transformed electrical signals, generate an OCT image based on the processed reflected light and an ultrasound image based on the processed electrical signals, and control display of a combined image based on the OCT image and the ultrasound image.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a schematic view of one example catheter of an intravascular ultrasound imaging system, in accordance with this disclosure.

FIG. 2 is a block diagram illustrating an example control unit that may be used to implement various techniques of this disclosure.

FIG. 3 is a schematic longitudinal cross-sectional view of one example of an imaging core that may be used to implement various techniques of this disclosure.

FIG. 4 is a schematic longitudinal cross-sectional view of another example of an imaging core that may be used to implement various techniques of this disclosure.

FIGS. 5A and 5B are conceptual images of a combined optical coherence tomography and ultrasound image, in accordance with this disclosure.

FIG. 6 is a flow diagram illustrating an example method for imaging patient tissue, in accordance with the disclosure.

### DETAILED DESCRIPTION

In general, this disclosure describes techniques that combine intravascular ultrasound (“IVUS”) and Optical Coherence Tomography (“OCT”) into a single catheter that utilizes a micro-motor driven imaging core for imaging of patient tissue, e.g., a blood vessel wall. Images produced using OCT systems generally have a higher resolution (sometimes referred to as in-vivo histology) than images produced using IVUS systems. OCT systems, however, generally cannot penetrate as deeply into tissue as IVUS systems.

In many applications it would be advantageous to combine IVUS and OCT capabilities into a single catheter. Using the techniques of this disclosure, IVUS and OCT are integrated into a single catheter that utilizes a micro-motor driven imaging core. One advantage to this design is a small package that incorporates IVUS and one or more optical fibers.

Another advantage to the techniques described in this disclosure is that the use of a micro-motor in the imaging core reduces Non-Uniform Rotational Distortion (NURD) from both the OCT image and the IVUS image. NURD arises when a rotating drive shaft extends the length of a catheter shaft of a catheter that has passed through twists and turns of a blood vessel system. In this setting, rotation at a tip of the catheter can be unsteady. That is, the catheter may not rotate at a uniform speed during its revolution, and the catheter may not remain fixed at one location in the vessel as it rotates. Using the techniques of this disclosure, a micro-motor is located within the imaging core at the distal end of the catheter, thereby locating the drive shaft at the distal end of the catheter rather than extending the length of the catheter shaft.

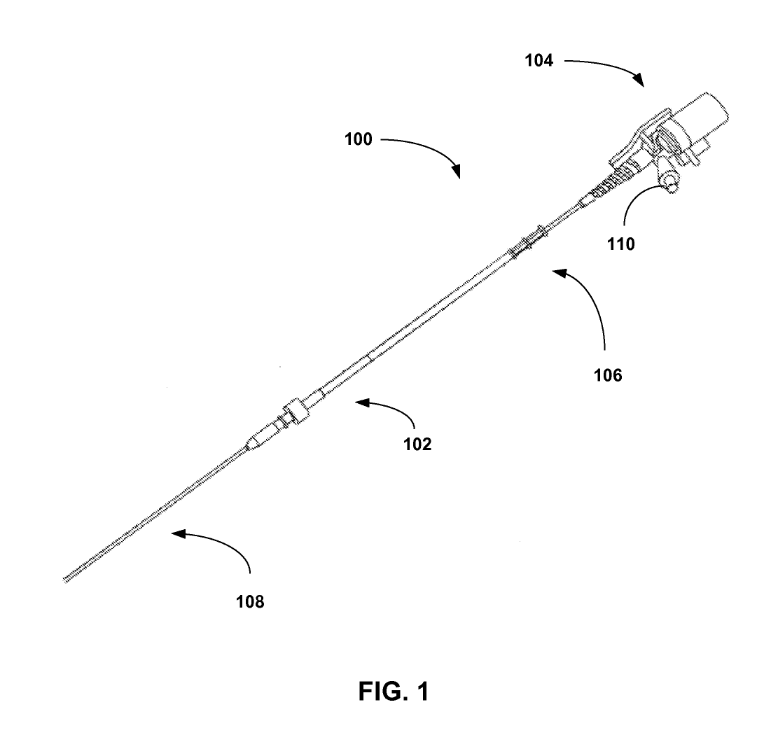

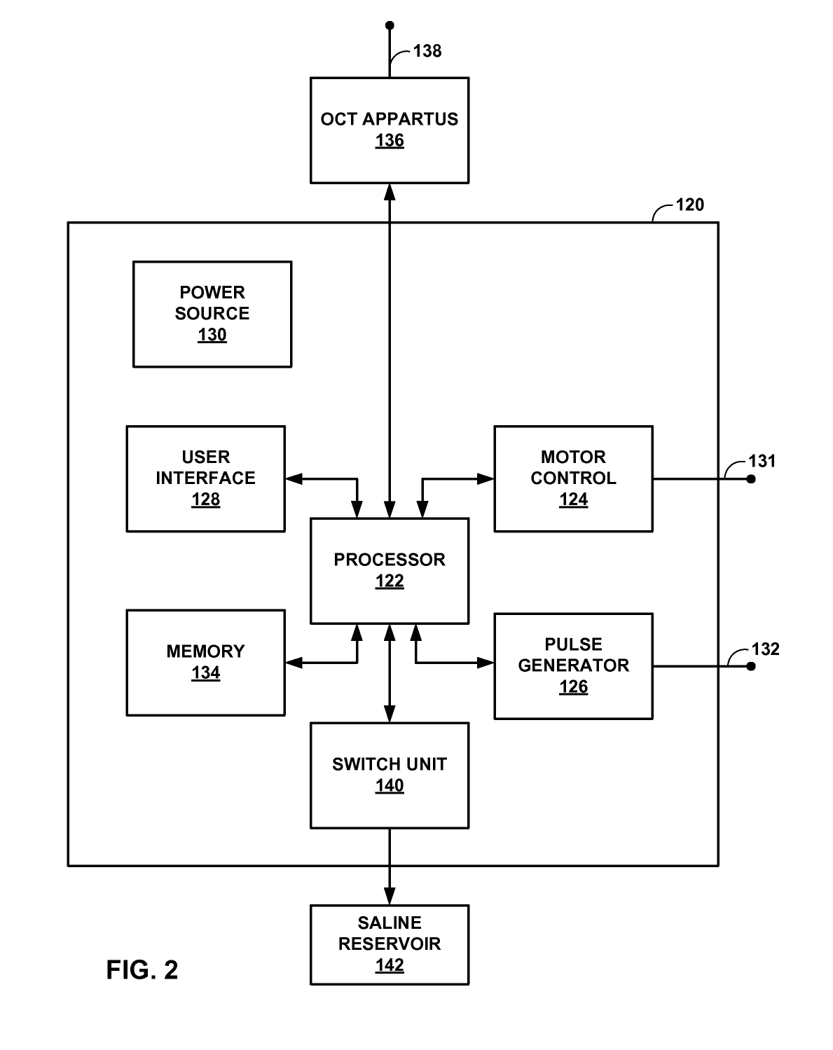

FIG. 1 is a schematic view of one example catheter that may be used to implement various techniques of this disclosure. As seen in FIG. 1, a catheter, shown generally at 100, includes elongated member 102 and hub 104. Elongated member 102 includes proximal end 106 and distal end 108. Proximal end 106 of elongated member 102 is coupled to hub 104, and distal end 108 of elongated member 102 is configured and arranged for percutaneous insertion into a patient. In at least some example implementations, catheter 100 defines one or more flush ports, such as flush port 110. In one example, flush port 110 is defined in hub 104. In some examples, hub 104 is configured and arranged to couple to a control unit (shown in FIG. 2). In some example configurations, elongated member 102 and hub 104 are formed as a unitary body. In other examples, elongated member 102 and catheter hub 104 are formed separately and subsequently assembled together.

FIG. 2 is a block diagram illustrating an example control unit that may be used to implement various techniques of this disclosure. In the example configuration depicted in FIG. 2, control unit 120 includes processor 122 that controls motor control unit 124, pulse generator 126, and user interface 128. In some examples, electric signals, e.g., pulses, transmitted from one or more transducers are received as inputs by processor 122 for processing. In one example, the processed electric signals from the transducer(s) are displayed as one or more images on a display of user interface 128.

Processor 122 can also be used to control the functionality of one or more of the other components of the control unit 120. In one example, processor 122 is used to control at least one of the frequency or duration of the electrical signals transmitted from pulse generator 126, a rotation rate of the imaging core by motor control unit 124, or one or more properties of one or more images formed on a display.

Processor 122 can include any one or more of a controller, a microprocessor, an application specific integrated circuit (ASIC), a digital signal processor (DSP), a field-programmable gate array (FPGA), or equivalent discrete or integrated logic circuitry. The functions attributed to processor 122 in this disclosure may be embodied as hardware, software, firmware, as well as combinations of hardware, software, and firmware.

Control unit 120 further includes power source 130. Power source 130 delivers operating power to the components of control unit 120. In one example, power source 130 includes a battery and power generation circuitry to generate the operating power.

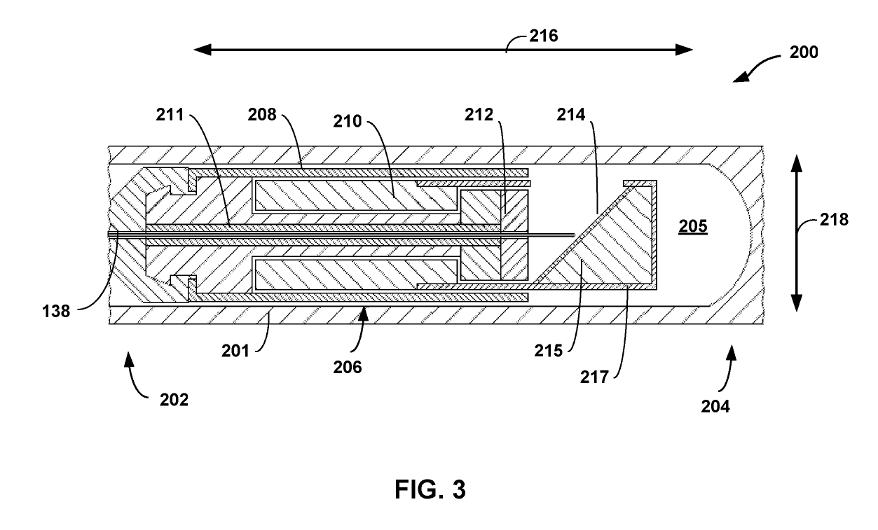

In addition, control unit 120 includes motor control unit 124. Motor control unit 124 supplies a current output to a motor (e.g., motor 206 in FIG. 3) in the imaging core of catheter 100 via one or more leads 131 in order to generate a magnetic field that rotates a reflective surface.

Pulse generator 126 generates electric signals, e.g., pulses, that are applied via one or more transducer conductors, e.g., leads 132, to one or more transducers (e.g., transducer 212 of FIG. 3) disposed in catheter 100. Leads 132 can be, for example, coaxial cable. User interface 128 includes a display (e.g., a touch screen display, liquid crystal display (LCD) monitor, or another display), and in some examples, includes a keyboard, and a peripheral pointing device, e.g., a mouse, that allows a user, e.g., clinician, to provide input to control unit 120.

Control unit 120 further includes memory 134. Memory 134 may include computer-readable instructions that, when executed by processor 122, cause processor 122 to perform various functions ascribed to control unit 120 and processor 122. The computer-readable instructions may be encoded within memory 134. Memory 134 may comprise computer-readable storage media such as a random access memory (RAM), read-only memory (ROM), non-volatile RAM (NVRAM), electrically-erasable programmable ROM (EEPROM), flash memory, or any other volatile, non-volatile, magnetic, optical, or electrical media.

As seen in FIG. 2, processor 122 is in communication with OCT apparatus 136. In some examples, OCT apparatus 136 is configured and arranged to generate an optical beam that is split into a reference light beam and a sample light beam directed onto a sample intended to be imaged. In some examples, OCT apparatus 136 includes one or more of the following: light source, beam splitter, diffraction grating, photodetector, and lens. In addition, in some examples, OCT apparatus 136 includes one or more filters for filtering a received signal, a demodulator for demodulating the signal, and an analog-to-digital (A/D) converter for converting the demodulated analog signal to a digital signal for processing by processor 122 of control unit 120. In one example, the filter(s), demodulator, and A/D converter are part of control unit 120. OCT apparatuses are well known to those of ordinary skill in the art and, for purposes of conciseness, will not be described in this disclosure in detail. More information regarding OCT can be found, for example, in U.S. Pat. Nos. 5,943,133, 7,426,037, and 7,415,303, the entire contents of each being incorporated herein by reference.

OCT apparatus 136 is in communication with one or more optical fibers 138 configured to guide the optical beams from a proximal end of the fiber to a distal end of the fiber. Optical fiber 138 extends through a catheter shaft lumen from a proximal end of the catheter toward a distal end of an imaging core.

In some example implementations, it is desirable to flush saline through the catheter to clear any blood out of the catheter prior to beginning an OCT scan. As such, in one example, control unit 120 includes switch unit 140 in communication with saline reservoir 142. Prior to transmission of an optical beam via OCT apparatus 136, processor 122 controls switch unit 140 to flush saline from saline reservoir 142 through a catheter lumen.

FIG. 3 is a schematic longitudinal cross-sectional view of one example of an imaging core that may be used to implement various technique of this disclosure. The imaging core, shown generally at 200 and contained within sheath 201, has proximal end 202 and distal end 204. Imaging core 200 is contained within a lumen, shown at 205, defined by sheath 201. In some example implementations, lumen 205 has an inner diameter of about 0.026 inches.

In accordance with certain techniques of this disclosure, imaging core 200 includes a micro-motor, shown generally at 206 (e.g., DC brushless motor, stepper motor, and the like), that includes a stator 208, rotor 210 (e.g., a rotating magnet), and stationary drive shaft 211. In one example implementation, stator 208 is configured as a three-phase winding. As seen in FIG. 3, drive shaft 211 of micro-motor 206 is contained within imaging core 200 rather than extending along the length of catheter 100. As mentioned above, utilizing a micro-motor, e.g., micro-motor 206, contained within imaging core 200 reduces NURD, thereby improving imaging quality.

Imaging core 200 further includes one or more transducers 212 configured and arranged for transforming applied electrical signals received from pulse generator 126 (FIG. 2) via lead(s) 132 (FIG. 2) to acoustic signals and also for transforming received echo signals to electrical signals. As seen in FIG. 3, in one example implementation, transducer(s) 212 are mounted at the distal end of stationary drive shaft 211. Although not depicted in FIG. 3, leads 132 extend through a drive shaft lumen defined by drive shaft 211 to control unit 120. In some example implementations, leads 132 are shielded electrical cables such as coaxial cable, twisted pair cable, and the like. In one example configuration, transducer 212 has a substantially circular cross-section that defines a hole at its center, thereby allowing leads 132 to extend through a lumen defined by drive shaft 211. In one example configuration, transducer 212 has a diameter of about 0.018 inches and defines a hole with a diameter of about 0.005 inches.

Control unit 120 is electrically connected to motor 206 via one or more leads 131 (FIG. 2). In particular, one or more leads 131 deliver current to stator 208 in order to generate a magnet field that rotates rotor 210. In at least one example configuration, leads 131 and leads 132 extend along at least a portion of the longitudinal length of the catheter 100.

Imaging core 200 further includes reflective surface 214, e.g., a mirror. In one example implementation, reflective surface 214 is engaged to absorbing backing material 215. In the example configuration depicted in FIG. 3, reflective surface 214 is coupled to magnet 210 via mirror holder 217 such that reflective surface 214 rotates along with magnet 210. As seen in FIG. 3, in some example configurations, reflective surface 214 is tilted at an angle that is not parallel with either a longitudinal axis 216 of imaging core 200 or diameter 218 of imaging core 200.

In some example implementations, reflective surface 214 is tilted at an angle so that acoustic signals output from transducer(s) 212, e.g., pulses of ultrasound energy, are reflected in a direction that is not parallel to longitudinal axis 216 of imaging core 200. In at least one example, reflective surface 214 is tilted at an angle so that acoustic signals output from transducers 212, e.g., pulses of ultrasound energy, are reflected toward patient tissue in a direction that is roughly perpendicular to the longitudinal length 216 of imaging core 200.

Reflective surface 214 is tilted at an angle so that at least some of the echo signals received from patient tissue (in response to the acoustic signals output from transducer(s) 212) are reflected to transducers 212. The echo signals are transformed into electric signals and transmitted to processor 122 for processing in order to produce an image. In at least some examples, reflective surface 214 is tilted at an angle so that at least some of the echo signals from patient tissue are reflected to a direction that is parallel to longitudinal axis 216 of imaging core 200.

In one example configuration, every other strip (not shown) in stator 208 is driven, while intervening strips are for structure, and are not electrically active. Three phase current is applied to three stator leads, causing the magnet and reflective surface 214 to rotate. Distal transducer 212 launches ultrasound pulses that reflect from reflective surface 214 into adjacent tissues.

Additional details regarding IVUS imaging systems may be found, for example, in the following references: U.S. Pat. Nos. 6,945,938 and 7,306,561; U.S. Patent Application Publication Nos. 2006/0100522; 2006/0253028; 2007/0016054; 2007/0003811; 2010/0249599; 2010/0249603; and 2010/0249604; and U.S. application Ser. Nos. 12/565,632 and 12/566,390, each of which is incorporated by reference herein in its entirety.

In accordance with the techniques of this disclosure, the imaging assembly that includes imaging core 200 further includes one or more optical fiber(s) 138 engaged to OCT apparatus 136. Optical fiber(s) 138 extend from the proximal end of catheter 100 (FIG. 1) through a lumen defined by drive shaft 211 (not shown) and through the hole defined by transducer 212. In one example, optical fiber(s) 138 are contained in a rigid tube and do not rotate, i.e., fiber(s) 138 are stationary. As seen in the example configuration depicted in FIG. 3, optical fiber(s) 138 extend from OCT apparatus 136 (FIG. 2) through the lumen defined by drive shaft 211 and terminate just proximal to reflective surface 214, e.g., a mirror tilted at 45 degrees. Both the ultrasound pulses and optical beams reflect from reflective surface 214 into adjacent tissue, e.g., blood vessel wall, in order to provide a circumferential view of the adjacent tissues as reflective surface 214 rotates.

Using the techniques of this disclosure, OCT images produced using OCT apparatus 136 are combined with ultrasound images produced using a micro-motor in order to generate an image that has better resolution than an ultrasound image and penetrates further into tissue than OCT. Utilizing the micro-motor reduces NURD in both the OCT image and the ultrasound image, resulting in a clearer image when the OCT image and the ultrasound image are combined. It should be noted that in some example implementations, the OCT images and the ultrasound images produced using the techniques of this disclosure can remain separated rather than combined into a single image.

In some examples, ultrasound pulses generated by transducer 212 cause reflective surface 214 to vibrate. As such, it is desirable in some instances to time the delivery of optical beams to reflective surface 214 such that any vibration caused by the ultrasound pulses is negligible. To that end, in one example implementation, optical beams necessary for OCT imaging are interleaved with the delivery and receipt of ultrasound pulses. Ultrasound pulses have a low duty cycle. A duty cycle is a ratio of a first time duration of an electric signal that is delivered, e.g., the ON time of an electric signal, to a second time duration, e.g., the period of the signal. As such, a duty cycle is defined by an ON time and an OFF time of a signal. In some examples, the ON time is when the signal is high and the OFF time is when the signal is low. For example, a signal that is ON for 1 second, e.g., high for 1 second, and OFF for 2 seconds, e.g., low for 2 seconds, has a duty cycle of ⅓ or 33%, i.e., the ratio of ON time to (ON time+OFF time). By way of specific example, processor 122 controls micro-motor 206 to rotate drive shaft 211 such that transducer 212 rotates at about 30 Hertz. In each revolution, 256 bursts of ultrasound are emitted, each burst lasting a few microseconds. As such, processor 122 controls pulse generator 126 such that a pulse is transmitted and a return pulse is received in about 10 microseconds, with about 120 microseconds between pulses. In this example, “delivery” of the signal includes transmission of the signal and receipt of the signal generated by the echo. Thus, the pulses are ON for about 10 microseconds and OFF for about 120 microseconds OFF, or a duty cycle of about 10/130.

In one example, processor 122 controls OCT apparatus 136 to continuously transmit and receive optical beams for OCT imaging, but blanks, or disregards, any data generated during a first time duration, e.g., the ON time of the electric signal delivered according to a duty cycle. Only data captured by OCT apparatus 136 during the OFF time of the electric signal is used to produce an image. In this manner, processor 122 generates the OCT image based on the processed reflected light received during the OFF time of the electric signal.

In another example, processor 122 controls pulse generator 126 to deliver electric signals to transducer(s) 212 according to a duty cycle, wherein the duty cycle is defined by an ON time and an OFF time of an electric signal, e.g., a signal generated by pulse generator 126, and wherein processor 122 controls transmission of an optical beam during the OFF time of the electric signal. In other words, optical beams for OCT images are interleaved with ultrasound pulses such that the optical beams are delivered and received only during the OFF time of the ultrasound pulses in order to minimize the chance that reflective surface 214 will still be vibrating at the time the optical beam is transmitted.

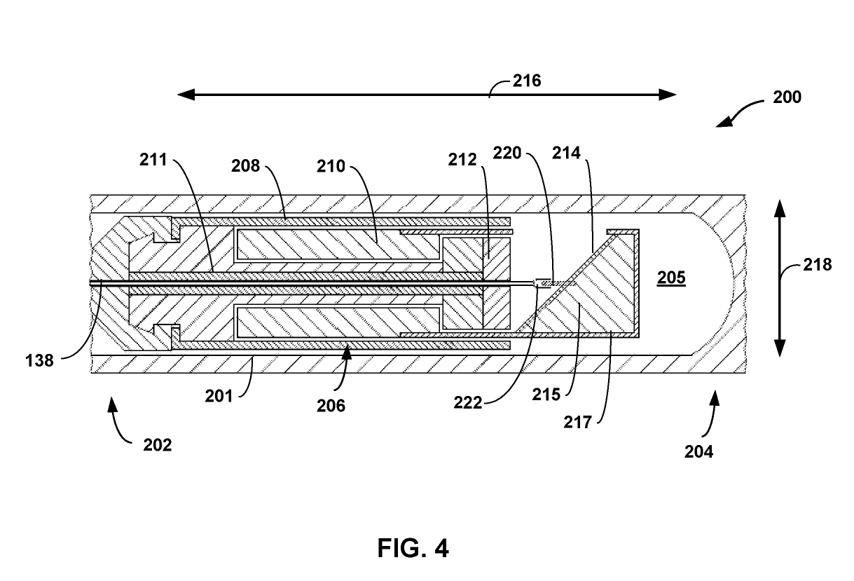

FIG. 4 is a schematic longitudinal cross-sectional view of another example of an imaging core that may be used to implement various techniques of this disclosure. In particular, FIG. 4 includes gradient index of refraction (“GRIN”) lens 220. In the example configuration depicted in FIG. 4, GRIN lens 220 focuses the optical beams transmitted via optical fiber(s) 138 at a right angle to the fiber(s) 138 as the beams exit the fiber(s). In this manner, GRIN lens 220 directs an optical beam transmitted via optical fiber(s) 138 instead of reflective surface 214. In one example, GRIN lens 220 includes a focusing mechanism and a prism.

As described above with respect to FIG. 3, micro-motor 206 and, in particular, rotor 210 rotates reflective surface 214 via mirror holder 217. GRIN lens 220 is attached to reflective surface 214 at a central axis and, as such, rotates with reflective surface 214. Because optical fiber(s) 138 do not rotate, i.e., fiber(s) 138 are stationary, imaging core 200 further includes rotating-to-stationary coupler 222.

In another example configuration, the imaging core, e.g., imaging core 200, includes a forward-focusing GRIN lens (not shown) mounted on the distal end of the optical fibers. In such a configuration, rather than direct the optical beams transmitted via the optical fibers at right angles to the fibers as the beams exit the fiber, the forward-focusing GRIN lens focuses the optical beams forward onto the reflective surface, e.g., reflective surface 214.

In this manner, certain techniques of this disclosure are directed to an imaging assembly and an imaging system. The imaging assembly includes a catheter, e.g., catheter 100, an imaging core with a micro-motor, e.g., imaging core 200, one or more transducer conductors, e.g., leads 132, an optical coherence tomography (OCT) apparatus, e.g., OCT apparatus 136, and one or more optical fibers, e.g., optical fibers 138, in communication with the OCT apparatus. The imaging system includes an imaging assembly, as described above, a user interface, e.g., user interface 128, and a control unit, e.g., control unit 120.

It should be noted that the micro-motor based techniques described throughout this disclosure can be applied to other optical fiber based technologies. For example, optical fiber based technologies such as fluorescence spectroscopy or photo-acoustic imaging can also be accommodated with the micro-motor based IVUS techniques described above in order to expand diagnostic capabilities.

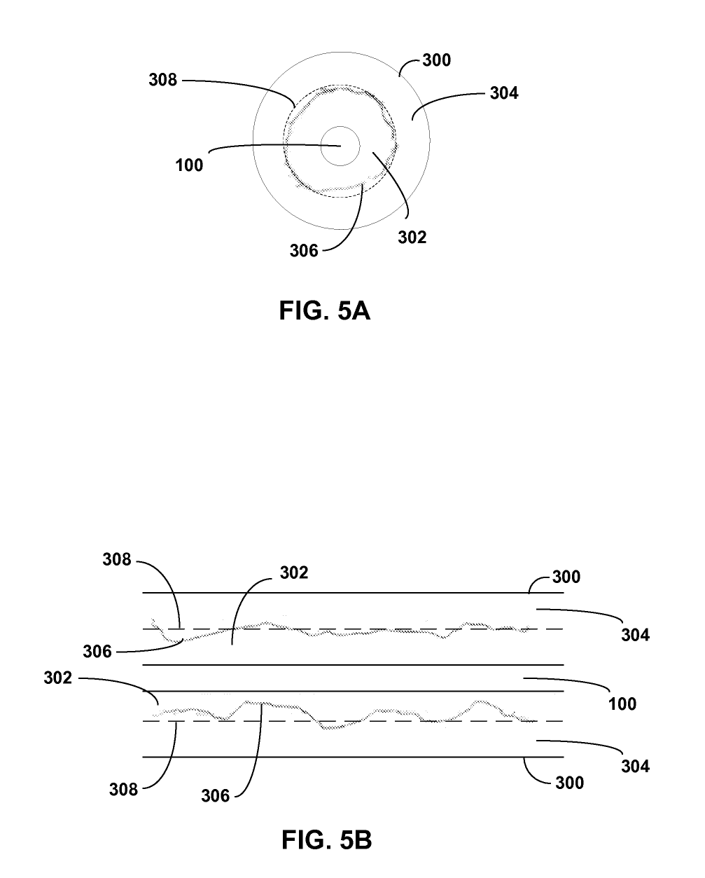

FIGS. 5A and 5B are conceptual images of a combined OCT and ultrasound image, in accordance with this disclosure. In particular, FIG. 5A is a conceptual image displaying a cross-sectional view of an artery having artery wall 300 using both OCT data and ultrasound data, and FIG. 5B is a conceptual image displaying a longitudinal view of artery wall 300 using both OCT data and ultrasound data. A catheter, e.g., catheter 100, is disposed within the artery.

As seen in FIGS. 5A and 5B, OCT data is displayed within OCT data region 302 for a first depth, e.g., about 1 millimeter, and ultrasound data is displayed within ultrasound data 304 for a second depth, e.g., greater than about 1 millimeter. The combined OCT and ultrasound data is shown at 306. In some example implementations, the boundary between OCT data 302 and ultrasound data 304, shown as dashed line 308, is determined by signal-to-noise ratios, e.g., when the signal-to-noise ratios are equal. Using various techniques of this disclosure, the OCT data region 302 and ultrasound data region 304 are coaxially aligned, as seen in FIGS. 5A and 5B. As such, the ultrasound and OCT images automatically align, without the need to co-register and shift side-by-side images.

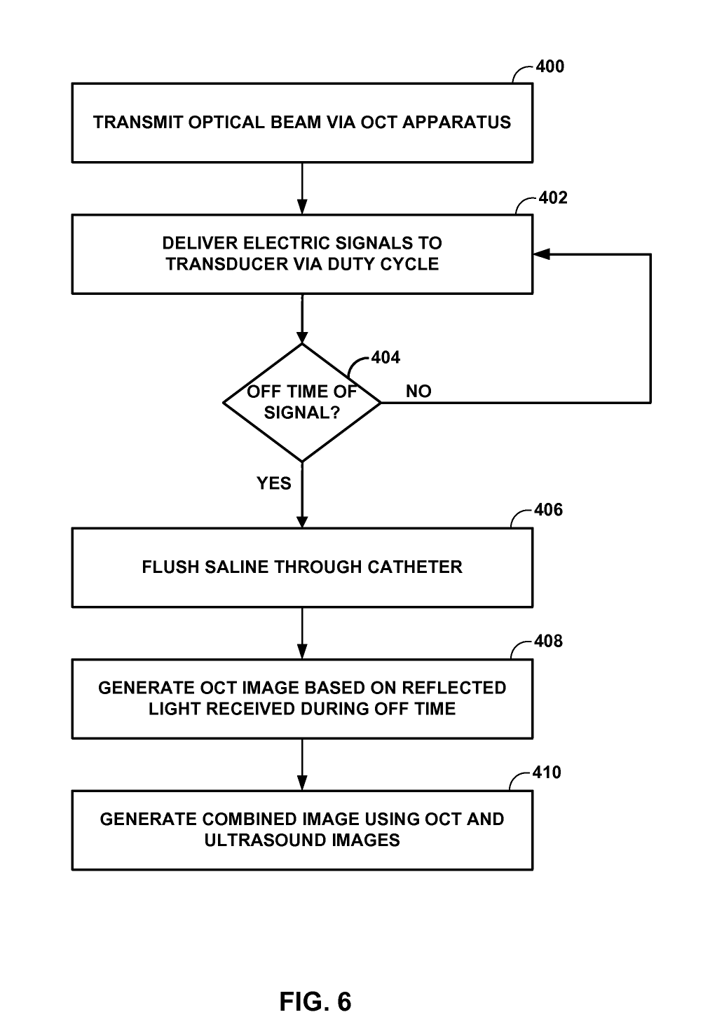

FIG. 6 is a flow diagram illustrating an example method for imaging patient tissue, in accordance with the disclosure. In FIG. 6, a processor, e.g., processor 122 of FIG. 2, controls transmission of an optical beam via an OCT apparatus, e.g., OCT apparatus 136 of FIG. 2, onto an area to be imaged, e.g., patient tissue, (400). In one example, the processor controls continuous transmission of the optical beam.

The processor controls delivery of electric signals, e.g., via pulse generator 126, to a transducer, e.g., transducer 212, according to a duty cycle, e.g., an ON time and an OFF time (402). If the signal is ON, e.g., high, (“NO” branch of block 404), then processor 122 continues to deliver the electric signal (406).

If the signal is OFF, e.g., low, (“YES” branch of block 404), then processor 122 controls a switch unit, e.g., switch unit 140, to flush saline from a saline reservoir, e.g., saline reservoir 142, through a catheter, e.g., catheter 100 (406). Processor 122 generates an OCT image based on the reflected light received from the area to be imaged, e.g., patient tissue, during the OFF time (408). Processor 122 generates a combined image using the generated OCT image and a generated ultrasound image (410).

Many examples of the disclosure have been described. These and other examples are within the scope of the following claims. Various modifications may be made without departing from the scope of the claims.

1. An imaging assembly comprising:
a catheter having a distal end and a proximal end, the catheter defining a catheter lumen extending from the proximal end to the distal end of the catheter, the catheter configured and arranged for insertion into the vasculature of a patient;
an imaging core having a distal end and a proximal end, the imaging core disposed in the distal end of the catheter lumen, the imaging core comprising
a drive shaft that extends from the proximal end of the imaging core toward the distal end of the imaging core, the drive shaft defining a shaft lumen,
a rotatable magnet disposed about the shaft lumen, the magnet configured and arranged to be rotatable by a magnetic field,
a reflective surface configured and arranged to rotate with the magnet, and
at least one transducer engaged to the drive shaft and positioned proximal to the reflective surface, the at least one transducer configured and arranged for transforming applied electrical signals to acoustic signals that are transmitted toward the reflective surface and also for transforming received echo signals to electrical signals, the at least one transducer defining a transducer lumen;
at least one transducer conductor electrically coupled to the at least one transducer and extending to the proximal end of the catheter;
an optical coherence tomography (OCT) apparatus configured to transmit an optical beam toward a tissue of the patient and receive reflected light from the tissue; and
at least one optical fiber in communication with the OCT apparatus that extends through the shaft lumen from the proximal end of the catheter toward the distal end of the imaging core through the transducer lumen, the optical fiber configured to guide the optical beam.

2. The assembly of claim 1, further comprising a graded index of refraction lens (GRIN), wherein the GRIN lens is fixedly engaged to the reflective surface and rotatably engaged to a distal end of the optical fiber.

3. The assembly of claim 1, further comprising a forward focusing lens, wherein the forward focusing lens is engaged to a distal end of the optical fiber.

4. The assembly of claim 1, further comprising:
an optical fiber tube that extends through the shaft lumen, the tube defining an optical fiber lumen, wherein the at least one optical fiber extends through the optical fiber lumen.

5. The assembly of claim 1, wherein the imaging core has a longitudinal axis, and wherein the reflective surface is tilted at an angle such that the acoustic signals transmitted from the at least one transducer toward the reflective surface reflect in a direction that is not parallel to the longitudinal axis of the imaging core.

6. The assembly of claim 1, further comprising a holder that is configured and arranged to secure the reflective surface to the magnet.

7. The assembly of claim 1, wherein the imaging core further comprises:
a stator disposed about the magnet, the stator having a plurality of windings, the plurality of windings configured and arranged to generate the magnetic field.

8. The assembly of claim 7, wherein the plurality of windings are configured and arranged in a three-phase winding geometry for receiving three-phase current.

9. The assembly of claim 8, wherein the stator receives the three-phase current via a control unit coupled to the imaging core, the control unit comprising:
a pulse generator electrically coupled to the at least one transducer via the at least one transducer conductor, the pulse generator configured and arranged to generate electric signals that are applied to the at least one transducer during a scan; and
a processor electrically coupled to the OCT apparatus and the pulse generator, the processor configured to:
control generation of the electric signals that are applied to the at least one transducer by the pulse generator;
control transmission of the optical beam, via the OCT apparatus, and delivery of the electric signals from the pulse generator to the at least one transducer;
process the received reflected light, via the OCT apparatus, and the transformed received echo signals;
generate an image based on the processed reflected light and echo signals; and
control display of the image.

10. An imaging system comprising:
the imaging assembly of claim 1;
a user interface; and
a control unit coupled to the imaging core, the control unit comprising:
a pulse generator electrically coupled to the at least one transducer via the at least one transducer conductor, the pulse generator configured and arranged to generate electric signals that are applied to the at least one transducer during a scan; and
a processor electrically coupled to the OCT apparatus and the pulse generator, the processor configured to:
control generation of the electric signals that are applied to the at least one transducer by the pulse generator;
control transmission of the optical beam, via the OCT apparatus, and delivery of the electric signals generated by the pulse generator to the at least one transducer;
process the received reflected light, via the optical coherence tomography circuitry, and the received transformed electrical signals;
generate an OCT image based on the processed reflected light and an ultrasound image based on the processed electrical signals; and
control display of a combined image based on the OCT image and the ultrasound image.

11. The system of claim 10,
wherein the processor controls delivery of the electric signals from the pulse generator to the at least one transducer according to a duty cycle,
wherein the duty cycle is defined by an ON time and an OFF time of the electric signals, and
wherein the processor controls transmission of the optical beam during the OFF time of the electric signals.

12. The system of claim 10,
wherein the processor controls delivery of the electric signals from the pulse generator to the at least one transducer according to a duty cycle,
wherein the duty cycle is defined by an ON time and an OFF time of the signals,
wherein the processor controls continuous transmission of the optical beam, and
wherein the processor generates the OCT image based on the processed reflected light received during the OFF time of the electric signals.

13. The system of claim 10, further comprising:
a switch unit; and
a saline reservoir comprising saline,
wherein the processor configured to control transmission of the optical beam, via the OCT apparatus, is further configured to:
control operation of the switch unit in order to flush saline from the reservoir through the catheter lumen prior to transmission of the optical beam.

14. The system of claim 10, further comprising a graded index of refraction lens (GRIN), wherein the GRIN lens is fixedly engaged to the reflective surface and rotatably engaged to the optical fiber.

15. The system of claim 10, further comprising a forward focusing lens, wherein the forward focusing lens is engaged to a distal end of the optical fiber.
